(19) United States
(12) Patent Application Publication
(10) Pub. No.: US 2006/0282060 A1
(43) Pub. Date: Dec. 14, 2006

(54) Blood collecting device and method of fixing it

(76) Inventor: Ryoji Fujii, HIROSHIMA (JP)

Correspondence Address: WENDEROTH, LIND & PONACK, L.L.P., 2033 K STREET N. W., SUITE 800, WASHINGTON, DC, 20006-1021, US

(21) Appl. No.: 10/548,379

(22) PCT Filed: Mar. 31, 2004
(86) PCT No.: PCT/JP04/04748

§ 371 Date: Sep. 8, 2005

(30) Foreign Application Priority Data
Mar. 31, 2003 (JP) 52003-095575

(51) Int. Cl. A61B 19/00; A61B 5/00

(52) U.S. Cl. 604/403; 604/415; 600/573

(57) ABSTRACT

To provide a blood collecting attachment with which blood can be easily collected from a sampling port inserted in a blood flow path into a tube without using a syringe, a blood collecting instrument can be securely fixed to the sampling port, and accidental needlesticks can be prevented. The blood collecting attachment includes a tubular member one end of which is a needle part for piercing the tube and the other end of which is an insertion part to be inserted in the sampling port, and a fixing member for fixing the tubular member to the sampling port. The fixing member is joined with the tubular member so that the tubular member is joined with the tubular member so that the tubular member is fixed to or unfixed from the sampling port when a predetermined rotation operation is performed on the fixing member.

### TECHNICAL FIELD

The present invention relates to a blood collecting attachment which is used to collect blood from a sampling port connected with a blood flow path into a blood container. The invention also relates to a blood collecting device and method, and a method of fixing a blood collecting tubular member to the sampling port.

### BACKGROUND ART

A blood flow path for transferring blood to a patient from outside through tubing or the like is employed in treatments such as a blood transfusion and a dialysis. In these treatments, the need may arise to collect a blood sample from the blood flow path.

Conventionally, blood collection is conducted as follows.

A sampling line is provided in the blood flow path, and a pierceable plug (e.g. a rubber plug with no disc) is attached to an opening of the sampling line. To collect blood, the plug is pierced with a needle of a syringe or the like to draw the blood into the syringe, and then a cap of a vacuum blood collecting tube is pierced with the needle to convey the blood from the syringe into the vacuum blood collecting tube.

An injection port in which a luer of a syringe can be directly inserted has been developed recently. Blood collection can be conducted using such an injection port, too.

For example, Unexamined Japanese Patent Application Publications Nos. H11-197254 and 2002-95739 disclose an injection port which includes a disc valve having an insertion hole in the middle and a cover for holding the valve by covering its upper edges. A circular hole in which an insertion object can fit is formed at a center of the cover, to fix the insertion object to the injection port. When a male luer at a tip of a syringe is inserted into the injection port, the valve is opened by the push of the luer, which enables a fluid to be injected. When the luer is removed from the injection port, the valve closes on its own due to a restoring force.

Blood collection from such an injection port inserted in the above blood flow path into a vacuum blood collecting tube is performed in the following way. A tip of a syringe is inserted into the injection port to draw blood into the syringe. After this, a needle is attached to the syringe, and a cap of the vacuum blood collecting tube is pierced with the needle to transfer the blood from the syringe into the vacuum blood collecting tube.

Both of the above methods use a syringe. In general, a syringe is thrown away after being used once. Also, when handling a needle-equipped syringe or when attaching a needle to a syringe containing collected blood, a needlestick accident may occur due to an operational mistake or the like.

On the other hand, blood can be collected without using a syringe according to the following method. A holder equipped with a hollow needle, such as the one described in Unexamined Japanese Patent Application Publication No. 2000-60827, is attached to an injection port inserted in the blood flow path. When necessary, a cap of a blood collecting tube is pierced with the hollow needle to draw blood.

According to this method, the risk of accidental needlesticks can be reduced. However, since the holder is attached to the injection port by sticking a luer of the holder into the injection port, the connection between the holder and the injection port does not have high durability. Though the holder can be connected to the injection port with some stability by joining them together using tape or the like, it is still difficult to maintain stable connection between the holder and the injection port for a long time.

### DISCLOSURE OF THE INVENTION

The present invention aims to collect blood easily from a port inserted in a blood flow path into a blood collecting tube without using a syringe. The invention also aims to stably fix a blood collecting instrument to the port. The invention further aims to reduce the risk of accidental needlesticks.

The above aims can be achieved by a blood collecting attachment that is removably attachable to a sampling port inserted in a blood flow path, and is used to collect blood is from the sampling port into a container sealed with a pierceable cap, including: a tubular member one end of which is designed to pierce the cap of the container; and a fixing mechanism operable to fix the tubular member to the sampling port in a state where the blood is led from the sampling port to the other end of the tubular member.

In a state where this blood collecting attachment is attached to the sampling port, blood can be collected easily by piercing the cap of the container with the end of the tubular member when necessary. This eliminates the need for using a syringe and the need for inserting a luer into the port.

Here, the fixing mechanism may include: a fixing member operable to fix the tubular member to the sampling port in a state where the other end of the tubular member is connected to the sampling port, and when a predetermined operation is performed on the fixing member, unfix the tubular member from the sampling port.

According to this construction, the tubular member can be stably fixed to the sampling port. Which is to say, the tubular member is kept from disjoining from the sampling port even when a naturally-occurring external force acts upon the fixing member. This contributes to excellent connection durability.

The predetermined operation referred to here is preferably an operation with which the fixing member is acted upon by a force that differs from a naturally-occurring external force which can act upon the fixing member in a state where the blood collecting attachment is attached to the sampling port. The naturally-occurring external force is, for example, a force of pushing or pulling the fixing member toward the sampling port or in any other direction, and is generally linear. Also, the predetermined operation is preferably easy to perform by hand. For instance, the predetermined operation is an operation of exerting a nonlinear force on the fixing member, such as rotating, twisting, or pressing in a sandwiching state.

Also, according to the above construction, the container is directly linked to the blood flow path via the tubular member. This reduces the dead volume between the sampling port and the container at the time of blood collection.

Furthermore, the construction of the blood collecting attachment is relatively simple.

Here, the fixing member may include: an engaging part for engaging with the sampling port, and when the predetermined operation is performed on the fixing member, disengaging from the sampling port.

This enables the fixing member to unfix the tubular member from the sampling port.

Here, the fixing member may further include: a grip part on which an operator performs the predetermined operation, wherein the engaging part is joined to the grip part so as to move to disengage from the sampling port when the operator performs the predetermined operation on the grip part.

Here, the grip part may be provided around the tubular member.

This makes it easier for the operator to attach/remove the blood collecting attachment to/from the sampling port.

Here, the other end of the tubular member may be insertable into an opening of the sampling port.

This benefits stable connection in the case where the sampling port is equipped with a valve which covers an opening of the sampling port and a cover which holds the valve.

Here, an elastic sheath may cover the end of the tubular member.

This reduces the risk of accidental needlesticks. The sheath does not hamper the end of the tubular member from piercing the cap of the container at the time of blood collection, and returns to the original shape by its elasticity once the blood collection has finished. Hence the end of the tubular member is constantly kept from contact with the outside air.

Here, the blood collecting device may further include: a container holder which is removably attachable to the blood collecting attachment in a state where the container holder surrounds the end of the tubular member and can store the container.

This further enhances the accidental needlestick prevention effect. Also, since the container is held by the container holder at the time of blood collection, the blood collection can be carried out easily.

It should be noted that a fluid collecting attachment equivalent to the above blood collecting attachment can be attached to a sampling port inserted in a flow path of a medical fluid or the like, to collect a fluid into a container in the same way as above.

### BRIEF DESCRIPTION OF DRAWINGS

FIG. 1 is a perspective view showing outward appearances of a blood collecting port, a blood collecting attachment, a tube holder, and a blood collecting tube to which an embodiment of the invention relates.

FIG. 2 is a sectional view of the blood collecting port, the blood collecting attachment, and the tube holder shown in FIG. 1.

FIG. 3 shows a situation where an insertion part of the blood collecting attachment is inserted in an insertion hole of the blood collecting port.

FIG. 4 shows a mechanism whereby a fixing member of the blood collecting attachment is fixed to and unfixed from a cover of the blood collecting port.

FIG. 5 shows a situation where the blood collecting tube is inserted in the tube holder from an opening thereof and a cap of the blood collecting tube is pierced with a needle part of the blood collecting attachment.

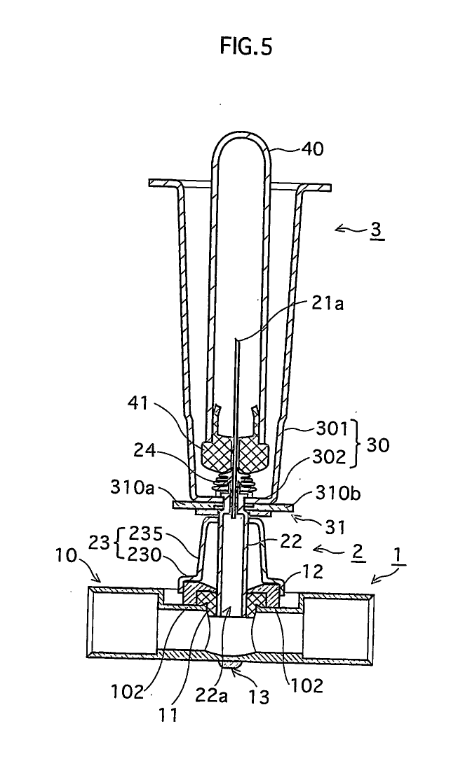

FIG. 6 shows an attachment according to a comparative example.

FIG. 7 shows modifications to the blood collecting attachment.

FIG. 8 shows a modification to the blood collecting attachment.

### BEST MODE FOR CARRYING OUT THE INVENTION

A device and method for collecting blood from a blood circuit according to an embodiment of the present invention are described below, by referring to the drawings.

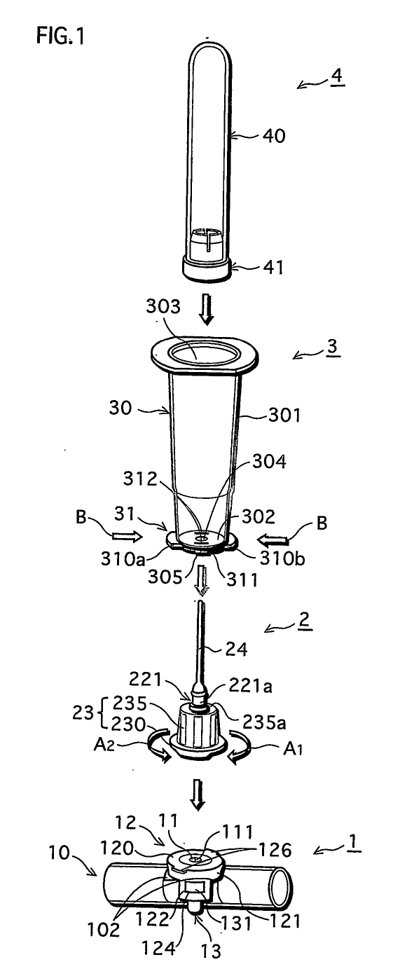

FIG. 1 is a perspective view showing outward appearances of a blood collecting port 1 inserted in a blood circuit, a blood collecting attachment 2 to be attached to the blood collecting port 1, a tube holder 3, and a blood collecting tube 4.

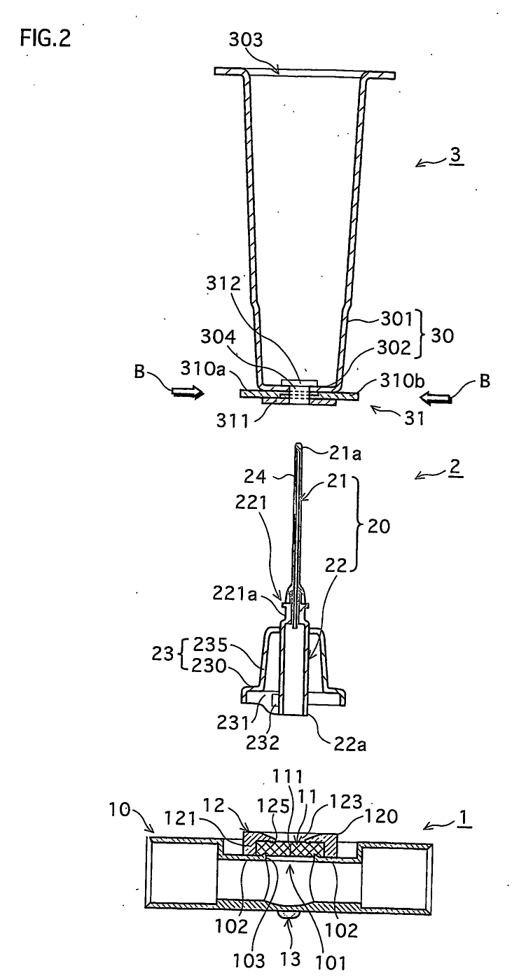

FIG. 2 is a sectional view of the blood collecting port 1, the blood collecting attachment 2, and the tube holder 3.

The blood collecting port 1, the blood collecting attachment 2, and the tube holder 3 shown in these drawings constitute a blood collecting device for collecting blood into the blood collecting tube 4.

The blood collecting tube 4 includes a container 40 having an opening and a cap 41 which seals the opening. The cap 41 is pierceable with a hollow needle. Blood can be sampled into the container 40 through the hollow needle piercing the cap 41.

The blood collecting tube 4 is typically a vacuum tube. In this case, the container 40 is a transparent glass tube inside of which is depressurized, and the cap 41 is a rubber cap pierceable with a needle of a syringe.

The blood collecting port 1 is inserted in a blood flow path for hemodialysis or the like. The blood collecting attachment 2 can be easily attached to and removed from the blood collecting port 1 by a predetermined operation performed by an operator, but will not easily disjoin from the blood collecting port 1 by a naturally-occurring external force which acts upon the blood collecting attachment 2. Likewise, the tube holder 3 can be easily attached to and removed from the blood collecting attachment 2 by a predetermined operation performed by the operator, but will not easily disjoin from the blood collecting attachment 2 by a naturally-occurring external force which acts upon the tube holder 3.

In this blood collecting device, the blood collecting attachment 2 and the tube holder 3 are constantly attached to the blood collecting port 1. When necessary, the cap 41 of the blood collecting tube 4 is pierced with a needle part 21a of the blood collecting attachment 2 to collect blood in the blood collecting tube 4.

The blood collecting port 1, the blood collecting attachment 2, and the tube holder 3 are each described in detail below.

(Blood Collecting Port 1)

As shown in FIGS. 1 and 2, the blood collecting port 1 includes a tubular port body 10 having a through hole 101 for blood collection on its tube wall, a disc valve 11 which blocks the through hole 101, a cover 12 which attaches the valve 11 to the port body 10, and a cover holder 13.

The port body 10 is cylindrical as a whole. Both ends of the port body 10 are connected with tubing of the blood flow path (not illustrated), so that blood flows through the port body 10. A flat-surface valve seat 102 for supporting the valve 11 and the cover 12 is formed in part of the tube wall of the port body 10. The valve seat 102 has an opening in the middle to provide the through hole 101.

For example, the port body 10 is formed of polycarbonate which has excellent transparency and high adhesiveness with tubes.

The valve 11 is disc-shaped and is formed of a rubbery elastic material (e.g. isoprene rubber) as a whole. A slit 111 is formed in the middle of the valve 11 so that a narrow tube can be inserted from the outside. The slit 111 may be shaped as a single straight line or three straight lines which radiate out in three directions from a center.

The cover 12 includes a ring-shaped top part 120 which covers upper edges of the valve 11 and presses them onto the valve seat 102, a cylindrical side part 121 which extends downward from the top part 120 so as to cover an entire side surface of the valve 11, and a pair of hanging parts 122 which extend downward from the side part 121 so as to sandwich the port body 10. An insertion hole 123 is provided in the middle of the top part 120 so that an insertion object can be inserted from the outside.

The cover 12 is an integral component formed of a material that is suitable for holding the valve 11 and the insertion object (e.g. polyacetal or polypropylene).

The cover holder 13 connects the pair of hanging parts 122 on the side of the port body 10 opposite to the valve seat 102. Projections 131 which fit in holes 124 formed in the pair of hanging parts 122 are formed at both ends of the cover holder 13.

A ring-shaped hook 125 is formed at inner edges of the top part 120 facing the insertion hole 123 toward the valve 11, whilst a ring-shaped groove in which the hook 125 is fit is formed on the surface of the valve 11. Likewise, a ring-shaped hook 103 is formed at inner edges of the valve seat 102 facing the through hole 101, toward the valve 11. The hook 103 and the inside surface of the top part 120 sandwich the valve 11, to seal between the valve 11 and the valve seat 102 around the through hole 101.

The cover 12 and the cover holder 13 are secured to the port body 10 from opposite sides, by the fitting of the projections 131 in the holes 124. In this state, the top part 120 of the cover 12 presses the valve 11 onto the valve seat 102.

Here, the hook 125 fits in the groove of the valve 11 and the hook 103 presses the valve 11 from the opposite side. This prevents misalignment between the valve 11 and the valve seat 102 and between the vale 11 and the top part 120, with it being possible to improve airtightness.

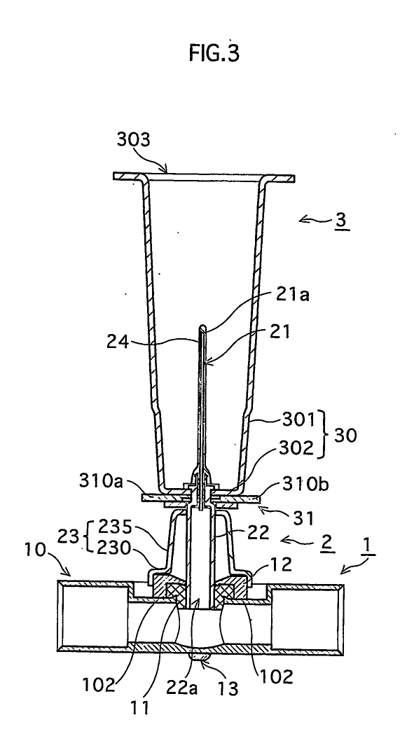

In this blood collecting port 1, when the insertion object is not inserted in the insertion hole 123, the slit 111 of the valve 11 is closed and therefore the valve 11 blocks the through hole 101 as shown in FIG. 2. When the insertion object is inserted in the insertion hole 123, on the other hand, the slit 111 is opened as shown in FIG. 3. Thus, only when an insertion part 22a of the blood collecting attachment 2 (described later) is inserted in the insertion hole 123, blood is introduced into the blood collecting attachment 2.

It should be noted here that the upper surface of the top part 120 gradually inclines downward toward the insertion hole 123, so as to guide the insertion object to the insertion hole 123.

Also, the cover 12 has a pair of projections 126 which project sideways from the top part 120, to engage the blood collecting attachment 2 with the blood collecting port 1.

This construction of the blood collecting port 1 is the same as that of a medical injection port described in Unexamined Japanese Patent Application Publication No. H11-197254.

(Blood Collecting Attachment 2)

The blood collecting attachment 2 includes a narrow tubular member 20 and a fixing member 23. The tubular member 20 has the needle part 21a for piercing the blood collecting tube 4 at an upper end, and the insertion part 22a to be inserted into the through hole 101 at a lower end. The fixing member 23 is used to fix the tubular member 20 to the blood collecting port 1.

The following describes an example where the tubular member 20 is formed by joining a needle tube 21 and a connecter tube 22 for linking the needle tube 21 to the through hole 101, although the whole tubular member 20 may instead be formed by a single needle tube.

An upper end of the needle tube 21 (i.e. the needle part 21a) is preferably formed of a high-strength material and provided with a sharpened tip, to be capable of piercing the cap 41.

The needle tube 21 may be formed of a metal. Alternatively, the needle tube 21 may be formed of a high-strength resin. When the needle tube 21 is formed of a resin, the resin may be strengthened by mixing it with a glass fiber or the like.

A needle sheath 24 is attached to the needle tube 21 so as to cover the needle part 21a. The needle sheath 24 may be formed of an elastic material (e.g. butyl rubber, isoprene rubber, or thermoplastic elastomer).

A lower end of the connector tube 22 (i.e. the insertion part 22a) is sized so as to be insertable in the insertion hole 123, the slit 111, and the through hole 101 of the blood collecting port 1. FIG. 3 shows a situation where the insertion part 22a is inserted in the insertion hole 123.

To join the connector tube 22 and the needle tube 21 together, a hole which corresponds to a size of the needle tube 21 is formed in an upper end 221 of the connector tube 22, and a lower portion of the needle tube 21 enters this hole. Here, the contact area of the upper end 221 and the needle tube 21 has a sufficient length to securely fix the needle tube 21 to the connector tube 22. Also, the upper end 221 and the lower portion of the needle tube 21 are bonded with an epoxy adhesive or the like.

A length of the connector tube 22 is preferably small to minimize the dead volume at the time of blood collection, in a range where the upper end 221 and the insertion part 22a can be provided.

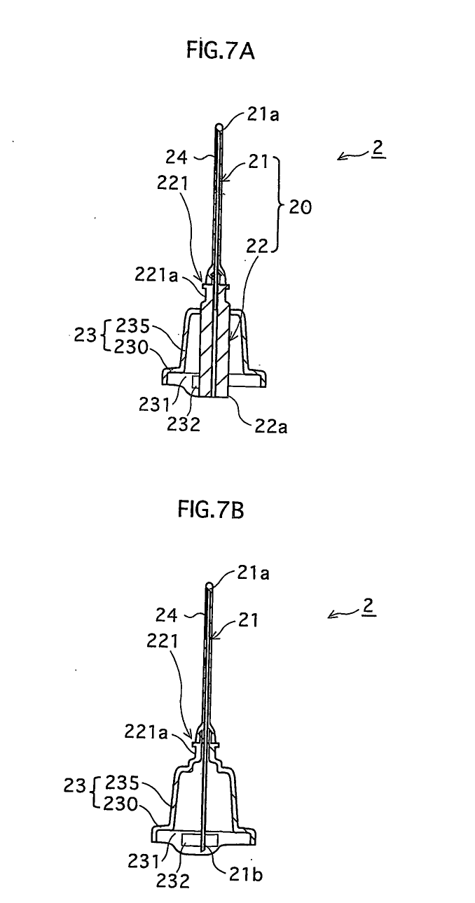

Also, an inside diameter of the connector tube 22 is preferably small to minimize the dead volume, in a range where blood can pass through. For example, the inside diameter of the connector tube 22 may be as small as an inside diameter of the needle tube 21, as shown in FIG. 7A. In this case, the valve 11 may be replaced with a rubber plug.

A tip of the upper end 221 is shaped as a hook for catching a lower end of the needle sheath 24, in order to keep the needle sheath 24 from becoming detached from the needle tube 21 due to a blood pressure inside the needle tube 21.

(Construction of the Fixing Member 23 and a Fixing/Unfixing Mechanism)

The fixing member 23 is fastened to the tubular member 20. Upon receiving a predetermined rotation operation, the fixing member 23 is fixed to or unfixed from the cover 12.

The following explains a detailed construction of the fixing member 23 and a fixing/unfixing mechanism.

The fixing member 23 includes a cover cap 230 which is placed over and engages with the cover 12, and a grip 235 which connects the cover cap 230 with the connector tube 22 and is shaped so as to be easily graspable by hand.

The grip 235 of the fixing member 23 and the connector tube 22 of the tubular member 20 are joined together, as a result of which the fixing member 23 is fastened to the tubular member 20. In more detail, an upper portion of the connector tube 22 penetrates an upper wall 235a of the grip 235, and the upper wall 235a and the connector tube 22 are bonded with an adhesive or the like.

The grip 235 has a smaller diameter than the cover cap 230, and projects upward from the cover cap 230. The fixing member 23 is shaped like a top hat as a whole.

The fixing member 23 can be formed using a resin (e.g. polypropylene or polycarbonate).

Note that the upper end 221 of the connector tube 22 is drawn so that the tube holder 3 can be easily attached. Also, a constricted part 221a which is to be held by a fixture mechanism 31 of the tube holder 3 (described later) is formed in the upper end 221 of the connector tube 22.

The following describes a mechanism whereby the fixing member 23 is fixed to or unfixed from the cover 12 by application of a rotation operation.

The cover cap 230 is ring-shaped and is designed to cover the upper surface of the top part 120 and an upper portion of the side part 121 of the cover 12. An inner surface of the cover cap 230 slides in contact with the surface of the cover 12, thereby rotating about the cover 12.

An inner side wall 231 of the cover cap 230 has depressions 232 in which the projections 126 fit when the cover cap 230 is placed over the cover 12 and rotated in a predetermined direction (indicated by hollow arrow A1 in FIG. 1).

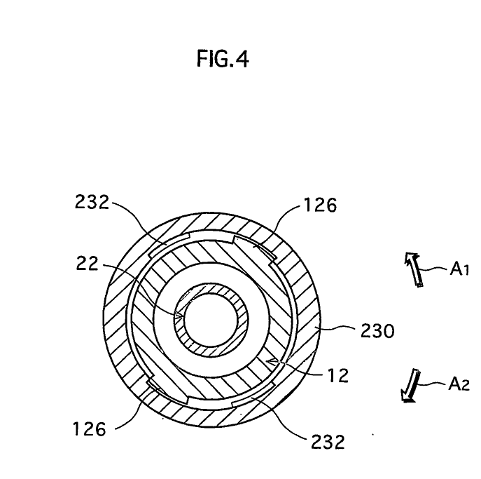

FIG. 4 is a sectional view of an upper portion of the cover 12 and the cover cap 230 in a state where the insertion part 22a is inserted in the insertion hole 123 of the blood collecting port 1 as shown in FIG. 3.

In FIG. 4, the projections 126 do not fit in the depressions 232. This being so, if the cover cap 230 is rotated in the direction of hollow arrow A1, the projections 126 fit in the depressions 232.

Once the projections 126 have fit in the depressions 232, the fixing member 23 is fixed to the cover 12. As a result, the blood collecting attachment 2 is kept from disjoining from the blood collecting port 1 even if a naturally-occurring external force acts upon the blood collecting attachment 2.

Since the grip 235 is provided around the tubular member 20, the operator can easily rotate the blood collecting attachment 2 about the tubular member 20 and attaches it to the blood collecting port 1 by turning the grip 235 by his or her hand.

On the other hand, when the cover cap 230 is rotated in a direction indicated by hollow arrow A2, the fixing member 23 is unfixed from the cover 12. In this way, the blood collecting attachment 2 can be removed from the blood collecting port 1.

(Tube Holder 3)

The tube holder 3 includes a tubular holder body 30 having a bottom surface in which the blood collecting tube 4 can be inserted, and the fixture mechanism 31 attached to the holder body 30 for removably attaching the holder body 30 to the blood collecting attachment 2.

The holder body 30 includes a tubular side wall part 301 and a bottom plate 302. An inside diameter of the side wall part 301 is a little larger than an outside diameter of the blood collecting tube 4 so as to accommodate the blood collecting tube 4.

A length of the side wall part 301 is larger than a length by which the needle tube 21 projects from the upper wall 235a, so that the tip of the needle part 21a does not reach an opening 303 of the tube holder 3. Meanwhile, the length of the side wall part 301 is smaller than that of the blood collecting tube 4, to ease the insertion and removal of the blood collecting tube 4 from the opening 303.

The bottom plate 302 has a through hole 304 in which the needle part 21a with the needle sheath 24 and the upper end 221 of the blood collecting attachment 2 can be inserted. The bottom plate 302 also has a pair of projections 305 which project from a bottom surface of the bottom plate 302 with the fixture mechanism 31 being interposed therebetween.

The holder body 30 is an integral component formed of a transparent or semi-transparent material (e.g. polyethylene, polypropylene, or polystyrene), to allow the operator to check the inside.

The fixture mechanism 31 is constructed as follows. In a state where the tube holder 3 is attached to the blood collecting attachment 2, the fixture mechanism 31 presses the constricted part 221a by sandwiching it. This pressure of the fixture mechanism 31 on the constricted part 221a is released when the operator presses the fixture mechanism 31 by sandwiching it.

The construction of the fixture mechanism 31 is explained in more detail below.

The fixture mechanism 31 is provided below the bottom plate 302. The fixture mechanism 31 includes a pair of pressing members 310a and 310b for holding the upper end 221 by sandwiching it, and a support member 311 for supporting the pair of pressing members 310a and 310b.

The support member 311 is a platelike member having a through hole (not illustrated) in which the needle part 21a and the upper end 221 can be inserted. A pair of engaging claws 312 are formed in the support member 311. The pair of engaging claws 312 engage with a pair of engaging holes formed in the bottom plate 302, as a result of which the support member 311 is attached to the bottom plate 302 in a parallel spaced relationship. The pair of pressing members 310a and 310b are slidably held between the bottom plate 302 and the support member 311.

The constricted part 221a of the upper end 221 is positioned so as to adjoin to the pair of pressing members 310a and 310b when the needle part 21a and the upper end 221 are inserted in the through hole 304 until the upper wall 235a of the grip 235 becomes adjacent to the support member 311 of the fixture mechanism 31.

Though not illustrated, an energizing means impels the pair of pressing members 310a and 310b to move away from each other. This being so, when inserted in the through hole 304, the constricted part 221a of the upper end 221 is pressed by the pair of pressing members 310a and 310b in a state of being sandwiched.

When the operator presses the pair of pressing members 310a and 310b by sandwiching them against the force of the energizing means (as indicated by hollow arrows B in FIGS. 1 and 2), the pressure on the constricted part 221a is released to unlock the tube holder 3 from the blood collecting attachment 2.

With the provision of this fixture mechanism 31, the tube holder 3 can be easily attached to the blood collecting attachment 2. Once attached, the tube holder 3 is kept from disjoining from the blood collecting attachment 2 even if a naturally-occurring external force acts upon the tube holder 3. On the other hand, the tube holder 3 can be easily removed from the blood collecting attachment 2 when the operator releases the lock.

This construction of the fixture mechanism 31 is the same as the one equipped in a tube holder described in Unexamined Japanese Patent Application Publication No. 2000-60827.

(Blood Collecting Method Using the Above Blood Collecting Device and its Effects)

In a state where the blood collecting attachment 2 is not attached to the blood collecting port 1 which is inserted in the blood circuit, the slit 111 of the blood collecting port 1 is closed and therefore blood in the blood circuit is isolated from the outside air.

The blood collecting attachment 2 and the tube holder 3 are attached to this blood collecting port 1 in the following manner.

To attach the blood collecting attachment 2 to the blood collecting port 1, the operator grasps the grip 235, inserts the insertion part 22a of the connector tube 22 into the insertion hole 123, and rotates the grip 235. Thus, the blood collecting attachment 2 is easily attached to the blood collecting port 1.

Once the blood collecting attachment 2 has been attached to the blood collecting port 1, the insertion part 22a of the connector tube 22 passes through the insertion hole 123 and the slit 111 and is connected with the blood flow path in the port body 10. As a result, blood is led from the blood flow path into the connector tube 22 and the needle tube 21. During this time, the connector tube 22 is supported by the insertion hole 123. Also, the projections 126 and the depressions 232 engage with each other, thereby fixing the blood collecting attachment 2 to the cover 12.

To attach the tube holder 3 to the blood collecting attachment 2, the operator inserts the needle part 21a with the needle sheath 24 and the upper end 221 of the blood collecting attachment 2 into the through hole 304 while pressing the pair of pressing members 310a and 310b, and then releases the grip from the pair of pressing members 310a and 310b. Hence the tube holder 3 is easily attached to the blood collecting attachment 2.

Once the tube holder 3 has been attached to the blood collecting attachment 2, the fixture mechanism 31 is fixed to the upper end 221. Also, the needle part 21a with the needle sheath 24 projects into an internal space of the holder body 30. Further, the pair of pressing members 310a and 310b hold the constricted part 221 by sandwiching it with the force of the energizing means, thereby locking the tube holder 3 to the blood collecting attachment 2. Accordingly, the tube holder 3 is kept from disjoining from the blood collecting attachment 2 even if a naturally-occurring external force acts upon the tube holder 3.

The operator can remove the tube holder 3 from the blood collecting attachment 2 simply by pressing the pair of pressing members 310a and 310b in a sandwiching state. Also, the operator can remove the blood collecting attachment 2 from the blood collecting port 1 simply by rotating the grip 235.

As described above, the blood collecting attachment 2 and the tube holder 3 can be easily attached to and removed from the blood collecting port 1. Also, the blood collecting attachment 2 and the tube holder 3 have favorable connection durability with the blood collecting port 1.

The connection durability of the blood collecting attachment 2 and the tube holder 3 is explained in detail below.

Suppose the blood collecting attachment 2 is attached to the blood collecting port 1. In this state, the blood collecting attachment 2 may be pushed or pulled toward the blood collecting port 1 or in an opposite or any other direction, due to a gravitational force or an environmental impactive force acting upon the blood collecting attachment 2. However, an external force which can detach the blood collecting attachment 2 from the blood collecting port 1 (i.e. a force of rotating the fixing member 23) is unlikely to act upon the blood collecting attachment 2. Likewise, suppose the tube holder 3 is attached to the blood collecting attachment 2. In this state, the tube holder 3 may be pushed or pulled toward the blood collecting attachment 2 or in an opposite or any other direction, due to a gravitational force or an environmental impactive force acting upon the tube holder 3. However, an external force which can detach the tube holder 3 from the blood collecting attachment 2 (i.e. a force of pressing the pair of pressing members 310a and 310b by sandwiching them) is unlikely to act upon the tube holder 3.

Therefore, the blood collecting attachment 2 and the tube holder 3 have favorable connection durability with the blood collecting port 1.

Furthermore, the above blood collecting device has the following effects.

In a state where the blood collecting attachment 2 and the tube holder 3 are attached to the blood collecting port 1, the tip of the needle part 21a is positioned below the opening 303 of the tube holder 3. This prevents accidental needlesticks. Also, the needle part 21a is covered with the needle sheath 24 and so is isolated from the outside air. This means the blood flowing into the needle part 21a will not be exposed to the outside air. This keeps the needle part 21a from being blocked by blood clotting and from being contaminated. In addition, the needle sheath 24 serves to prevent accidental needlesticks too.

To collect blood into the blood collecting tube 4, the blood collecting tube 4 is inserted into the tube holder 3 from the opening 303 so as to pierce the cap 41 with the needle part 21a. Here, the cap 41 is guided to the tip of the needle part 21a along the inner wall of the holder body 30, so that the cap 31 can be easily pierced with the needle part 21a.

As a result of piercing the cap 41 with the needle part 21a, the needle sheath 24 is compressed downward by the cap 41 and thereby removed from the needle part 21a. Hence part of the blood in the port body 10 flows through the connector tube 22 and the needle tube 21 into the blood collecting tube 3. In this way, the blood is connected. During this time, the blood collecting tube 4 is held by the tube holder 3. Accordingly, the operator does not need to hold the blood collecting tube 4 by his or her hand.

After the blood collection, the blood collecting tube 4 is removed from the needle part 21a. As a result, the compressed needle sheath 24 returns to the original shape to cover the needle part 21a.

Thus, while the blood collecting attachment 2 and the tube holder 3 are attached to the blood collecting port 1, the blood can be collected repeatedly according to need as described above. During when the blood is not being collected, the needle part 21a is covered with the needle sheath 24 so as not to be exposed to the outside air. This effectively suppresses the blockage of the needle part 21a, as explained above.

(Consideration on the Dead Volume at the Time of Blood Collection)

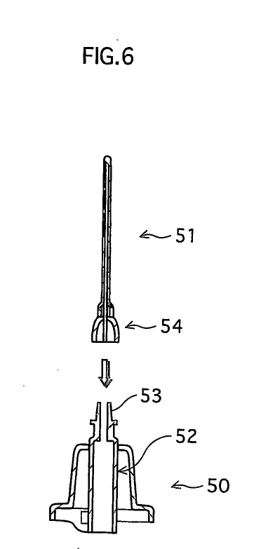

Since the blood collecting attachment 2 is constructed by directly joining the needle tube 21 to the connector tube 22, the dead space is small (that is, the dead volume at the time of blood collection is small). This is explained in detail below, using a comparative example shown in FIG. 6.

FIG. 6 shows an attachment 50 according to the comparative example. The attachment 50 is similar in construction to the blood collecting attachment 2 with the following exception. The attachment 50 does not include a needle tube 51. When necessary, the needle tube 51 is attached to a connector tube 52 in the attachment 50 by placing a root 54 of the needle tube 51 over a tip 53 of the connector tube 52. To do so, the tip 53 of the connector tube 52 is shaped as a luer, whereas the root 54 of the needle tube 53 is shaped as a cap.

Blood can be collected using this attachment 50 and needle tube 51, in the same way as when the blood collecting attachment 2 is used. However, when the attachment 50 and the needle tube 51 are used, the dead space is created at the tip 53. In contrast, the blood collecting attachment 2 effectively reduces the dead volume at the time of blood collection.

Besides, the needle tube 51 may disjoin from the attachment 50 if a naturally-occurring external force acts upon the needle tube 51. This causes lower connection durability of the needle tube 51 with the blood collecting port.

For these reasons, the blood collecting attachment 2 in which the needle tube 21 is directly joined to the connector tube 22 is favorable in terms of dead space reduction and connection durability.

When the blood collecting attachment 2 is not attached to the blood collecting port 1, a luer of a syringe can be inserted into the blood collecting port 1 to inject a medical fluid. Thus, the blood collecting port 1 serves as an injection port, too.

(Modifications)

The above embodiment describes the case where the needle part 21a is covered with the needle sheath 24, but the needle part 21a need not be covered with the needle sheath 24. When the needle part 21a is not covered with the needle sheath 24, the effect of suppressing the blockage and contamination of the needle part 21a may somewhat decrease. However, accidental needlesticks can be prevented so long as the tube holder 3 is attached to the blood collecting attachment 2.

The above embodiment describes the case where the tube holder 3 is attached to the blood collecting attachment 2, but the tube holder 3 may be omitted.

In this case, in a state where the blood collecting attachment 2 is attached to the blood collecting port 1, the needle part 21a with the needle sheath 24 is exposed to the outside. This decreases the effect of preventing accidental needlesticks. Besides, the operator needs to hold the blood collecting tube 4 at the time of blood collection. Nevertheless, the risk of accidental needlesticks can still be reduced to some extent, since the needle part 21a is covered with the needle sheath 24.

The following describes modifications to the fixing member 23 of the blood collecting attachment 2 and to the fixture mechanism 31 of the tube holder 3.

A mechanism whereby the fixing member 23 is fixed to and unfixed from the cover 12 by a rotation operation is not limited to that of the above embodiment. For instance, a male screw and a female screw which correspond to each other may be formed respectively in the cover 12 and the fixing member 23.

Also, though the above embodiment describes the case where the fixing member 23 is fixed to and unfixed from the cover 12 by a rotation operation, the invention is not limited to such. For example, a mechanism whereby the fixing member 23 is fixed to and unfixed from the cover 12 by a twisting operation or by an operation of pressing in a sandwiching state may be used instead.

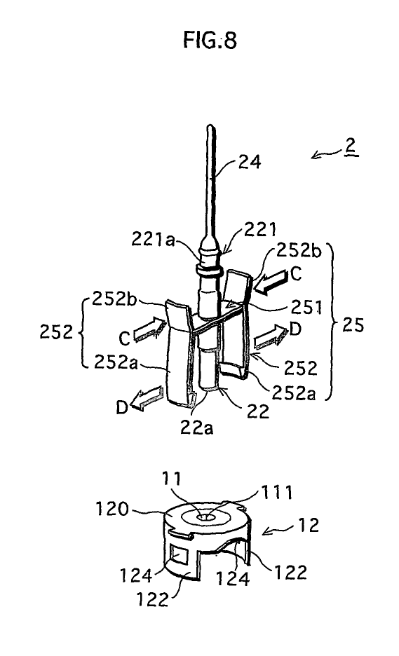

FIG. 8 shows an example of this modification. In the drawing, a fixing member 25 having a clip mechanism is joined to the connector tube 22, instead of the fixing member 23 which is fixed to and unfixed from the cover 12 by a rotation operation.

This fixing member 25 includes a support part 251 attached to the connector tube 22, and a pair of rodlike swing parts 252 which are swingably supported by the support part 251. The fixing member 25 is formed of a deformable elastic material.

The pair of swing parts 252 are supported by the support part 251 interposed therebetween. The cover 12 is sandwiched by lower portions of the pair of swing parts 252. The lower portions of the pair of swing parts 252 have engaging parts 252a which fit in the holes 124 of the pair of hanging parts 122. Also, portions of the pair of swing parts 252 above the support part 251 function as a pair of grips 252b. When the operator presses the pair of grips 252b in a direction indicated by hollow arrows C, the engaging parts 252a open in a direction indicated by hollow arrows D.

To attach this blood collecting attachment 2 to the blood collecting port 1, the operator presses the pair of grips 252b by sandwiching them to open the engaging parts 252a. In this state, the insertion part 22a is inserted in the slit 111 and the engaging parts 252a are fitted in the holes 124. In so doing, the blood collecting attachment 2 can be easily fixed to the cover 12. To remove the blood collecting attachment 2 from the blood collecting port 1, the operator presses the pair of grips 252b by sandwiching them to open the engaging parts 252a. This releases the lock. In this state, the insertion part 22a is removed from the slit 111. In so doing, the blood collecting attachment 2 can be easily removed from the cover 12.

As an alternative, the fixing member 23 may be formed of an elastic material, with the upper surfaces of the projections 126 being inclined. In this case, the blood collecting attachment 2 can be attached to the blood collecting port 1 simply by pushing the blood collecting attachment 2 into the blood collecting port 1, and removed from the blood collecting port 1 by a rotation operation.

The fixture mechanism 31 can be modified in the same way as the fixing member 23. Which is to say, a male screw and a female screw that correspond to each other may be formed respectively in the constricted part 221a and the fixture mechanism 31.

Also, the contact areas of the pressing members 310a and 310b with the constricted part 221a may be inclined. In so doing, the tube holder 3 can be attached to the blood collecting attachment 2 simply by pushing the tube holder 3 into the blood collecting attachment 2, and removed from the blood collecting attachment 2 by pressing the pressing members 310a and 310b by sandwiching them.

The above embodiment describes the case where the needle tube 21 and the connector tube 22 are joined together to form the tubular member 20 and the needle tube 21 is connected to the through hole 101 via the connector tube 22. Alternatively, a lower end of the needle tube 21 may be directly connected to the through hole 101 without using the connector tube 22.

FIG. 7B shows an example of this modification. In the drawing, the connector tube 22 except the constricted part 221a is omitted, and the needle tube 21 is extended downward so that its lower end 21b reaches the through hole 101. Also, the fixing member 23 is directly joined to the constricted part 221a or the needle tube 21. In this case, a rubber packing without a slit may be provided in the blood collecting port 1 in place of the valve 11. When this blood collecting attachment 2 is attached to the blood collecting port 1, the lower end 21b of the needle tube 21 pierces the rubber packing to reach the through hole 101.

By such directly connecting the lower end 21b of the needle tube 21 to the blood collecting port 1, the dead space of the blood collecting attachment 2 can be further reduced.

The above embodiment describes the case where the blood collecting port 1 in which the through hole 101 is sealed with the valve 11 is used. However, the above blood collecting method can equally be achieved when a blood collecting port in which the through hole 101 is not sealed with a valve but opened is used, by attaching the blood collecting attachment 2 and the tube holder 3 to such a blood collecting port.

When the through hole 101 is opened, the connector tube 22 can be connected to the blood flow path in the port body 10 without the need for the insertion part 22a.

Also, the above blood collecting method can equally be achieved when a three-way cock is inserted in the blood flow path instead of a blood collecting port, by fixing an equivalent of the cover 12 to a branch opening of the three-way cock and attaching an equivalent of the blood collecting attachment 2 (i.e. an attachment in which a fixing member is joined to a tubular member) to the cover.

The above embodiment describes the case where the blood collecting port 1 is inserted in the blood flow path and blood is drawn from the blood flow path, but this is not a limit for the invention. A fluid other than blood can equally be collected into a container by attaching an equivalent of the blood collecting attachment 2 to a sampling port inserted in a medical fluid flow path or the like.

### INDUSTRIAL APPLICABILITY

The invention can be used to collect blood from a sampling port inserted in a blood flow path into a container. The invention can also be used to collect a fluid other than blood from a sampling port inserted in a medical fluid flow path or the like into a container.

1. A blood collecting attachment that is removably attachable to a sampling port inserted in a blood flow path, and is used to collect blood from the sampling port into a container sealed with a pierceable cap, comprising:
a tubular member one end of which is designed to pierce the cap of the container; and
a fixing mechanism operable to fix the tubular member to the sampling port in a state where the blood is led from the sampling port to the other end of the tubular member.

2. The blood collecting attachment of claim 1,
wherein the fixing mechanism includes:
a fixing member operable to fix the tubular member to the sampling port in a state where the other end of the tubular member is connected to the sampling port, and when a predetermined operation is performed on the fixing member, unfix the tubular member from the sampling port.

3. The blood collecting attachment of claim 2,
wherein the fixing member includes:
an engaging part for engaging with the sampling port, and when the predetermined operation is performed on the fixing member, disengaging from the sampling port.

4. The blood collecting attachment of claim 3,
wherein the predetermined operation is a nonlinear operation.

5. The blood collecting attachment of claim 4,
wherein the fixing member further includes:
a grip part on which an operator performs the predetermined operation, and
the engaging part is joined to the grip part so as to move to disengage from the sampling port when the operator performs the predetermined operation on the grip part.

6. The blood collecting attachment of claim 5,
wherein the grip part is provided around the tubular member.

7. The blood collecting attachment of claim 2,
wherein the fixing member is swingably attached to the tubular member and forms a clip mechanism.

8. The blood collecting attachment of claim 2,
wherein the other end of the tubular member is insertable into an opening of the sampling port.

9. The blood collecting attachment of claim 1,
wherein an elastic sheath covers the end of the tubular member.

10. A blood collecting device comprising:
a sampling port inserted in a blood flow path; and
the blood collecting attachment of claim 1.

11. The blood collecting device of claim 10 further comprising:
a container holder which is removably attachable to the blood collecting attachment, in a state where the container holder surrounds the end of the tubular member and can store the container.

12. The blood collecting device of claim 11,
wherein the tubular member is formed by connecting a hollow needle and a tube so as to communicate with each other, and
the container holder includes:
a fixture mechanism operable to fix to a connecting portion of the hollow needle and the tube.

13. A blood collecting device comprising:
a sampling port inserted in a blood flow path; and
the blood collecting attachment of claim 3,
wherein the sampling port includes:
an engaged part for engaging with the engaging part, and
the engaging part disengages from the engaged part when the predetermined operation is performed on the fixing member.

14. A blood collecting device comprising:
a sampling port including (a) a port body which is inserted in a blood flow path and has an opening, (b) a valve which covers the opening and has a slit, and (c) a cover which holds the valve; and
the blood collecting attachment of claim 8.

15. A method of collecting blood from a sampling port inserted in a blood flow path into a container sealed with a cap, comprising:
a step of preparing a tubular member one end of which is designed to pierce the cap of the container, and fixing the tubular member to the sampling port in a state where the blood is led from the sampling port to the other end of the tubular member; and
a step of piercing the cap of the container with the end of the tubular member.

16. A method of fixing a blood collecting tubular member one end of which is designed to pierce a cap of a container, to a sampling port inserted in a blood flow path, comprising:
a step of joining a fixing member to the tubular member; and
a step of fixing the tubular member to the sampling port by engaging the fixing member with the sampling port in a state where blood is led from the sampling port to the other end of the tubular member.
